(19) United States
(12) Patent Application Publication
(10) Pub. No.: US 2020/0138638 A1
(43) Pub. Date: May 7, 2020

(54) COMPACT TAMPON APPLICATOR

(71) Applicant: ALYK, Inc., New York, NY (US)

(72) Inventors: Maria Eugenia Ferraro, New York, NY (US); Monica Belsito, New York, NY (US); Jochen Rainer Schaepers, New York, NY (US)

(73) Assignee: ALYK, Inc., New York, NY (US)

(21) Appl. No.: 16/664,810

(22) Filed: Oct. 26, 2019

Related U.S. Application Data
(60) Provisional application No. 62/754,443, filed on Nov. 1, 2018.

(51) Int. Cl. A61F 13/26; A61F 13/20; A61F 13/34

(52) U.S. Cl. CPC A61F 13/266; A61F 13/34; A61F 13/2097

(57) ABSTRACT

A tampon applicator.

This application claims the benefits of U.S. Provisional Patent Application No. 62/754,443 filed on Nov. 1, 2018, the contents of which are incorporated herein by reference.

### FIELD OF THE INVENTION

The present invention relates to tampon applicators and particularly tampon applicators that provide a compact and unobtrusive form that is easy to manufacture and use.

### BACKGROUND OF THE INVENTION

Numerous designs for tampon applicators that introduce tampons or pledgets intravaginally have been disclosed in the art and many are currently sold commercially. Most tampon applicator designs employ an outer open ended tubular holder or barrel that holds a tampon or pledget within the inner tubular cavity and a plunger adapted to move within the inner tubular cavity of the outer barrel to expel the tampon or pledget from the outer barrel. The plunger is also generally tubular, though smaller in diameter than the inner tubular cavity of the outer barrel, and is telescopically positioned therein so that by moving the plunger into one end of the outer barrel, the tampon or pledget may be ejected from the opposite end. Many of these prior and commercially available designs assemble the tampon applicator by placing the plunger and tampon or pledget into the top or insertion tip of the outer barrel and then subjecting the assembled structure to further processing to close the insertion tip. Examples of various tampon applicator designs can be found in U.S. Pat. Nos. 3,895,634; 4,291,696; 5,533,966; 6,019,744; 6,432,075; 6,773,422; 7,704,242; 8,372,027; 8,444,590; 9,192,522; 9,339,419 and 9,737,443.

Although the various prior art tampon applicators work for their intended function, i.e., the insertion and placement of a tampon or pledget into the vagina, there is always a need to improve upon the prior designs. In particular there is a need for a compact and easy to manufacture tampon applicator that is also comfortable and easy to use.

### SUMMARY OF THE INVENTION

The foregoing need is met by the present invention, which is a compact tampon applicator that is easy to manufacture and easy to use.

More specifically, the present invention is a tampon applicator comprising an outer barrel with an insertion end and a gripping end opposite the insertion end, a tampon or pledget within the inner cavity or inner portion of the outer barrel, and a plunger which pushes the tampon or pledget through the inner cavity of the outer barrel and out of the inner cavity of the outer barrel through the insertion end of the outer barrel.

In one embodiment the plunger is a folded or segmented plunger with a first portion of the plunger located within the inner cavity (inside) of the outer barrel near the gripping end of the applicator prior to use and a second portion of the plunger located outside the inner cavity of the outer barrel prior to use. The first portion of the plunger has a pledget contact end that is inside the gripping end of the outer barrel and will contact the base of the pledget inside the outer barrel. The first portion of the plunger will also have a connection end that is opposite the pledget contact end and connected to the second portion of the plunger. The second portion of the plunger has a connection end that is connected to the connection end of the first portion of the plunger, and a push end that is opposite the connection end. During storage and/or prior to use, the second portion of the plunger is located outside the inner cavity of the outer barrel with the connection end near the gripping end of the barrel and the push end near the insertion end of the outer barrel. In preparation for use, the push end of the second portion of the plunger is moved away from the insertion end of the outer barrel preferably in a radial manner to a position that creates a continuous longitudinal axis between the first portion of the plunger and the second portion of the plunger with the second portion of the plunger extended away from the gripping end of the outer barrel. Once the second portion of the plunger is in a continuous longitudinal axis orientation with the first portion of the plunger, the user can exert pressure on the push end of second portion of the plunger towards the insertion end of the outer barrel and slide the pledget contact end of the first portion of the plunger into contact with the pledget (if not already in contact) and push the pledget through the insertion end of the outer barrel.

In another embodiment of the present invention, the plunger is substantially encompassed by the outer barrel during storage or prior to use and the pledget is substantially encompassed by the plunger which is substantially encompassed by the outer barrel during storage or prior to use. In this embodiment, the outer barrel has an insertion end and a gripping end opposite the insertion end. The plunger has an insertion end and a push end that is opposite the insertion end. The plunger is sized to fit within the interior of the outer barrel with the insertion end of the plunger located near the insertion end of the outer barrel prior to use and the push end of the plunger located near the gripping end of the outer barrel. The push end of the plunger extends away from the gripping end of the outer barrel. In preparation for use, the plunger is moved from a nested position wherein a substantial portion of the plunger is encased within the outer barrel, by pulling the push end of the plunger away from the gripping end of the outer barrel along a longitudinal axis of the outer barrel and plunger to a ready to use position. Once the plunger is in the ready to use position, the insertion end of the plunger engages the base of the pledget that is opposite the insertion tip of the pledget. Once the insertion end of the plunger engages the base of the pledget, the user can exert pressure on the push end of the plunger towards the insertion end of the outer barrel and push the pledget through the insertion end of the outer barrel.

In a further embodiment, the present invention is a tampon applicator comprising an outer barrel with an insertion end and a gripping end opposite the insertion end, a tampon or pledget within the inner cavity (inside) of the outer barrel, and a plunger which pushes the tampon or pledget through the inner cavity of the outer barrel and out of the inner cavity of the outer barrel through the insertion end of the outer barrel wherein the applicator can be easily manufactured by forming the outer barrel with a desired shape and design at the insertion end, inserting the tampon and/or the pledget into the gripping end of the outer barrel opposite the preformed insertion end. The tampon or pledget may be inserted separately or simultaneously with the plunger.

### BRIEF DESCRIPTION OF THE DRAWING

FIGS. 1A to 1G show embodiments of a tampon applicator of the present invention wherein FIG. 1A shows the individual parts of an embodiment, FIG. 1B shows side views of the assembled applicator in a storage or prior to use mode, FIG. 1C shows a side view of the assembled applicator in a ready to use mode, FIG. 1D shows a top view of the assembled applicator in a storage or prior to use mode, FIG. 1E shows side cross sectional views taken along line A-A of FIG. 1D of the assembled applicator in various stages of the use mode including the final stage of the use mode wherein the pledget has been pushed through the insertion tip of the outer barrel and FIGS. 1F and 1G shows alternative embodiments of the plunger shown in FIG. 1A.

FIGS. 2A to 2H show another embodiment of a tampon applicator of the present invention wherein FIG. 2A shows the individual parts of the embodiment, FIGS. 2B, 2C and 2D show side views of the assembled applicator in a storage or prior to use mode, FIG. 2E shows a side view of the assembled applicator in a ready to use position, FIG. 2F shows the assembled applicator after the pledget has been pushed from the insertion tip, FIG. 2G shows a top view of the assembled applicator and FIG. 2H shows a side cross sectional view of the assembled applicator in a storage or prior to use mode taking along line A-A of FIG. 2G.

FIGS. 3A to 3C show a further embodiment of a tampon applicator of the present invention, FIG. 3A shows a side view of the assembled applicator in a storage or prior to use mode, FIG. 3B shows a side view of the assembled applicator in a ready to use position, FIG. 3C shows the assembled applicator after the pledget has been pushed from the insertion tip.

### DETAILED DESCRIPTION OF THE INVENTION

The tampon applicator of the present invention may be made by any means commonly known in the art such as injection molding, preferably by injection molding the outer barrel in a single injection molded piece and injection molding the plunger in a single injection molded piece or in multiple injection molded pieces.

The outer barrel and plunger may be made of any material that may be injection molded or commonly used for tampon applicators such as thermoplastics, cellulose based materials, biopolymers, etc. Examples of materials that may be used include but are not limited to polyolefins such as polyethylene and polypropylene (including polyolefin copolymers); polyesters such as polyethylene terephthalate; polyamides such as nylon; polyurethanes; polystyrene; polycaprolactone; polyvinyl alcohol; ethylene-vinyl acetate copolymers; elastomers such as silicones, natural rubbers, and synthetic rubbers including block copolymers; cellophane; cellulose materials such as hydroxypropyl methylcellulose and hydroxypropyl cellulose, starches, polyethylene oxides and combinations thereof. Examples of some of these materials can be found in U.S. Pat. Nos. 9,339,580, 6,773,422 and 5,910,520 and International Patent Application No. WO 2002/07352 which are incorporated herein by reference.

The pledget employed in the tampon applicator of the present invention may be made of any absorbent material having acceptable absorbency and modulus of elasticity properties capable of absorbing and/or retaining liquid. The absorbent structure can be manufactured in a wide variety of sizes and shapes and from a wide variety of liquid-absorbing materials. It is, of course, desirable to use absorbent materials having a minimum content of extraneous soluble materials since the product may be retained in the body for a considerable period of time, i.e., absorbent materials that contain little or preferably no unnecessary soluble matter which could dissolve and enter the body. Retained soluble extraneous materials could cause a safety hazard if they are toxic, irritant, or sensitive. A representative, non-limiting list of useful absorbent materials includes cellulosic materials, such as rayon, cotton, hemp, cannabis, wood pulp, creped cellulose wadding, tissue wraps and laminates, peat moss, and chemically stiffened, modified, or cross-linked cellulosic fibers; synthetic materials, such as polyester fibers, polyolefin fibers, absorbent foams, e.g., a flexible resilient polyurethane foam, absorbent sponges, super-absorbent polymers, absorbent gelling materials, formed fibers, such as capillary channel fibers and multi limbed fibers, synthetic fibers, or any equivalent material or combinations of materials, or mixtures of these. Preferably the pledget is made of 100% organic cotton.

The pledget may be formed in any known conventional shape. Preferably the pledget has a rounded or tapered insertion tip and a substantially flat end or base opposite the insertion tip. The pledget may have a smooth outer surface or it may have a plurality of grooves or recesses. The grooves or recesses may be continuous or discontinuous and run in any direction or pattern on the outer surface of the pledget. For example, the pledget may employ a plurality of continuous grooves that run longitudinally from the insertion tip to the base of the pledget or a plurality of continuous groves that run in a spiral pattern from the insertion tip to the base.

Representative examples of pledget that may be employed in the present invention can be found in U.S. Pat. Nos. 8,864,732; 8,777,916; 6,310,269 and 5,911,712 which are incorporated herein by reference.

The pledget should also comprise a withdrawal string or thread. The withdrawal string may be formed of any materials commonly known in the art, but is preferably formed from 100% organic cotton.

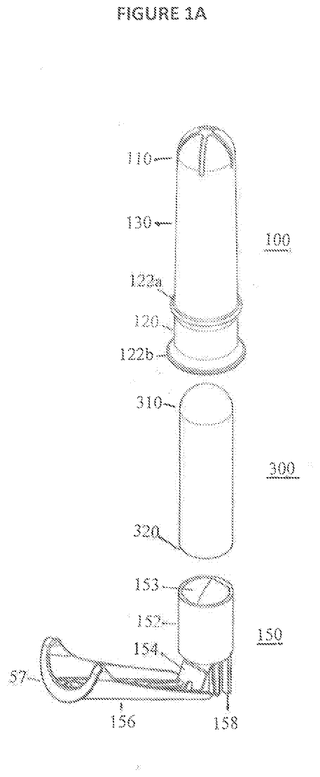

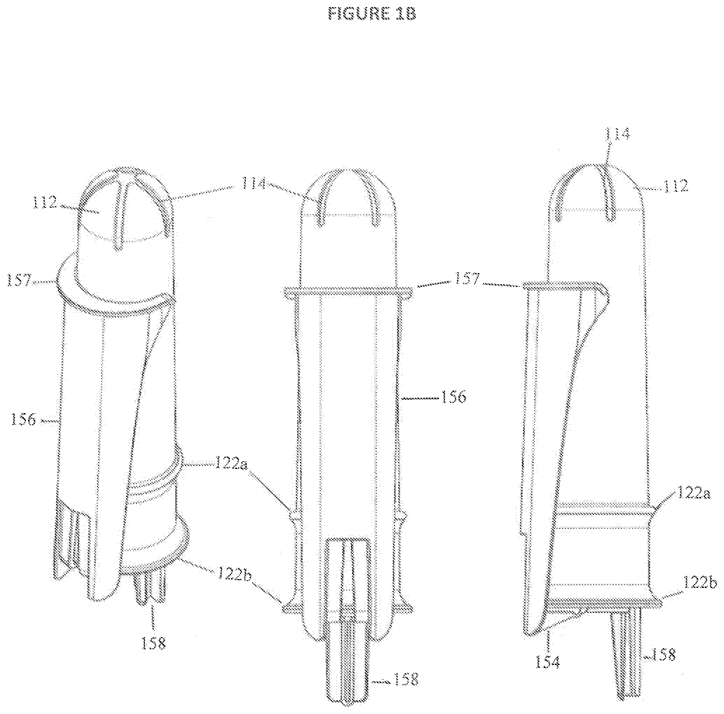

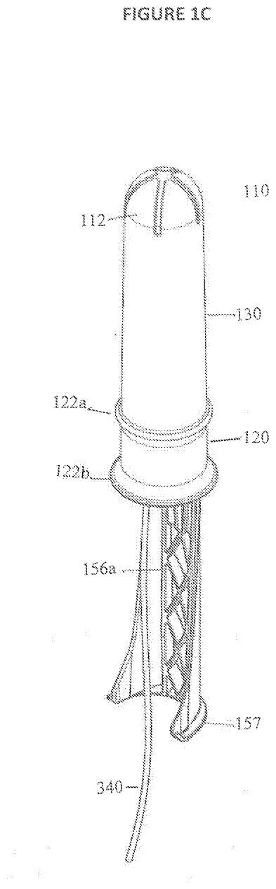

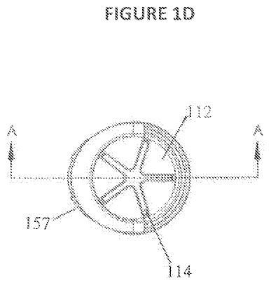

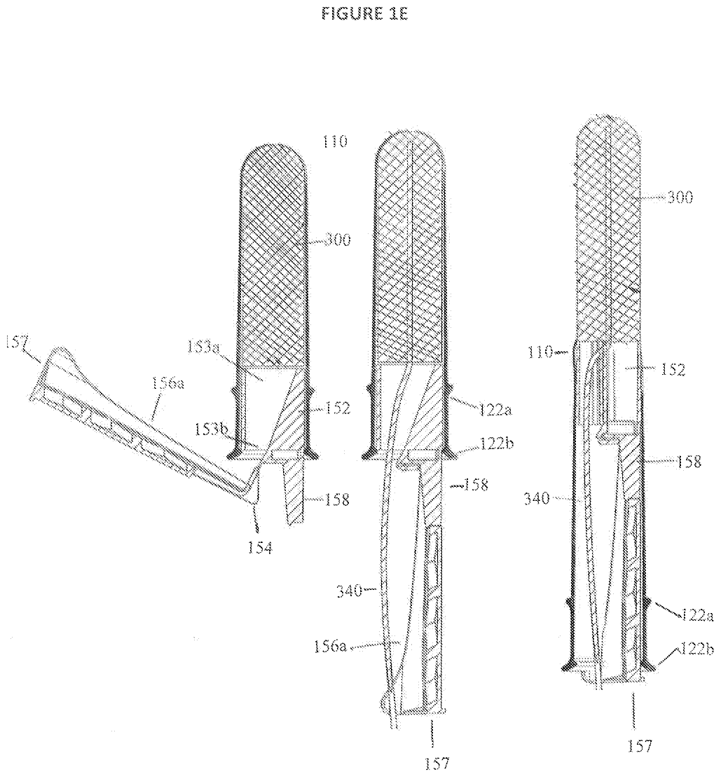

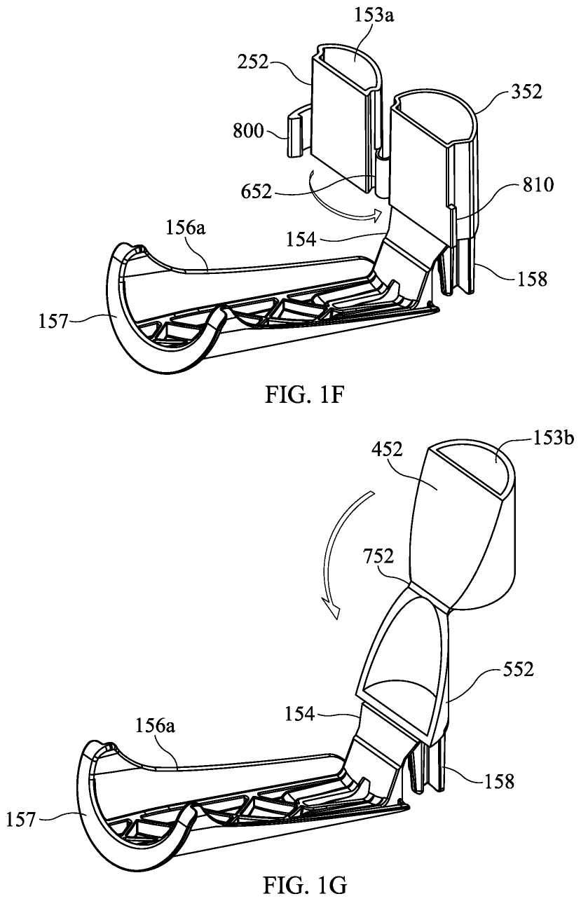

Referring now to the drawings, FIGS. 1A to 1E depict one embodiment of the present invention wherein the plunger is segmented with a portion of the plunger external to the outer barrel prior to use. More specifically, FIG. 1A shows the three primary components of the segmented plunger embodiment which comprise the outer barrel 100, pledget 300, and segmented plunger 150. The outer barrel 100 comprises an insertion tip 110, a gripping region 120 that is opposite the insertion tip 110, a main body region 130 located between the insertion tip 110 and gripping region 120, and an inner cavity that encases the pledget 300 and a portion of the segmented plunger 150.

As shown in FIGS. 1A-1C, the insertion tip 110 may be domed or hemispherical in shape or it may be tapered or bullet shape to allow easier and more comfortable insertion into the vaginal cavity during use. The domed and tapered shapes are well known in the art and described in detail in numerous references such as U.S. Pat. Nos. 3,895,634; 6,432,075 and 6,773,422 which are incorporated herein by reference. The insertion tip 110 comprises a plurality of petals 112 that are formed into the insertion tip and separated by a plurality of cuts, slits, or spacings 114. The petals 112 may be sized and shaped to provide protection to the pledget 300 prior to use and during insertion, i.e., use of the tampon applicator, into the vaginal cavity. The petals 112 may also be sized and shaped to protect the user from the rough surfaces of the pledget 300 during insertion. Embodiments of the present invention may comprise 3 to 8 petals, preferably 4 to 6 petals. The dimensions of the petals 112 are also known in the art and described in numerous references such as U.S. Pat. Nos. 3,895,634; 6,432,075 and 6,773,422 which are incorporated herein by reference. In certain embodiments, the petals 112 form the dome or tapered shape of the insertion tip 110 and the dome or tapered shape is preferably formed during the manufacture of the outer barrel 100. In one embodiment the dome or tapered shape of the insertion tip 110 is formed during the injection molding of the outer barrel 100 and before the pledget 300 is loaded into the outer barrel 100. Alternatively, the dome or tapered shape of the insertion tip 110 can be formed after initial forming of the outer barrel 100 and after the pledget 300 has been loaded into the outer barrel 100. The preforming of the dome or tapered shape of the insertion tip 110 during the manufacture of the outer barrel 100 and/or before the insertion of the pledget 300 into the outer barrel is the preferred method because it allows for a simpler manufacturing.

The gripping region 120 is located on the outer surface of the outer barrel 100 and opposite the insertion tip 110. The gripping region 120 is an area of the outer barrel 100 which will allow the user to grip or hold the tampon applicator during use. The gripping region 120 may comprise one or more structures to improve the user's hold on the outer barrel 100. Many of the grip-improving structures are known in the art and may include one or more indentations into the outer barrel 100, one or more raised projections from the outer barrel 100, varying surface roughness or textures compared to the main body 130 and insertion tip 110, or a combination thereof. If indentations or projections are employed in the gripping region 120, they may be continuous around the gripping region 120 or discontinuous. In one embodiment, the gripping region 120 comprises at least one, preferably at least two, raised projection 122 that extends outwardly from the outer surface of the outer barrel 100 and defines the gripping region 120 of the outer barrel. At least one of the raised projections 122a should be placed on the outer surface of the outer barrel 100 between the insertion tip 110 and the terminal end of the outer barrel 100 that is opposite the insertion tip 110 and preferably at a distance from the terminal end of the outer barrel 100 that is sufficient to accommodate the user's fingertip, preferably the middle and/or thumb fingertip. In certain embodiments, the distance from one of the raised projections 122a to the terminal end of the outer barrel 100 is about 10 to 25 mm. In another embodiment, a second raised projection 122b may be employed at or near the terminal end of the outer barrel 100 that is opposite the insertion tip 110. In this embodiment, first raised projection 122a and the second raised projection 122b define the gripping region 120. As shown in FIG. 1A to FIG. 1C the raised projections 122a and 122b extend outwardly from the outer barrel 100 in a continuous and uniform plane. In an alternative embodiment, the first raised projection 122a may be a continuous undulating or sinusoidal projection wherein the peaks and troughs of the projection in relation to the second raised projection 122b aid the user to grip the tampon applicator in the correct location for use. For example, the area between the high peak of the sinusoidal projection 122a and the second projection 122b will define an area that can accommodate a user's fingertip while the area between the trough of the sinusoidal projection 122a and the second projection 122b will define an area that cannot accommodate a user's fingertip. This pattern will thereby aid the user in properly gripping the tampon applicator.

In some embodiments, the terminal end of the outer barrel 100 that is opposite the insertion tip 110 and/or the raised lower projection 122b is flat or planar. Alternatively, the terminal end of the outer barrel 100 that is opposite the insertion tip 110 and/or the raised lower projection 122b is a concave shape, i.e., curving inwardly or upwardly toward the insertion tip 110. This concave shape may also aid the user in correctly orienting their fingers in the gripping region 120.

The pledget 300 comprises an insertion tip 310 and a base 320 that is opposite the insertion tip 310. The insertion tip 310 may be domed or tapered in a shape similar to the shape of the insertion tip 110 of the outer barrel. In some embodiments, the insertion tip 310 of the pledget is similar to the shape of the insertion tip 110 of the outer barrel 100. In this embodiment, prior to and/or during use of the tampon applicator, a portion of the petals 112 of the insertion tip 110 will be in contact with and supported by the insertion tip 310 of pledget 300. The pledget 300 also has a removal string 340 that will extend from the pledget 300, preferably the base 320 of pledget 300, and extend through a passageway, opening, channel or cavity of segmented plunger 150 and/or the inner cavity of outer barrel 100. The pledget 300 is stored in the inner cavity of the outer barrel 100 and is preferably loaded into the inner cavity from the terminal end of the outer barrel 100 that is opposite the insertion tip 110. The loading of the pledget 300, preferably after the dome or tapered shape of the insertion tip 110 has been created, may occur prior to or concurrently with the addition of the plunger to the outer barrel 100. The loading of the pledget 300 through the terminal end or gripping region of the outer barrel 100 with a preformed domed or tapered insertion tip shape also allows the inner surface of the outer barrel 100 to be formed without the need for retaining and/or support structures such as barbs or hooks in the insertion tip region of the outer barrel.

The segmented plunger 150 is a foldable or segmented plunger comprising a first plunger portion 152 that will be fully or partially located inside the outer barrel 100, i.e., within the inner cavity of the outer barrel 100, near the gripping region 120 and a second plunger portion 156. The first plunger portion 152 should be sized and shaped to allow the segmented plunger 150 to telescopically move within the inner cavity of the outer barrel 100. Alternatively, the first plunger portion 152 should be sized to slidably fit within the inner cavity of the outer barrel 100. In certain embodiments the first plunger portion 152 is cylindrical, semi-, hemi-, or partially cylindrical with a maximum diameter that is less than the diameter of the inner cavity of the outer barrel 100.

The segmented plunger 150 may also comprise one or more hinges 154 that will allow the first plunger portion 152 and the second plunger portion 156 to be formed in a single mold piece and subsequently configured to form the segmented plunger 150 that may be slidably located within the inner cavity of the outer barrel 100 and used to push the pledget 300 through the insertion tip 110 of the outer barrel 100. Alternatively, the segmented plunger 150 may be formed in two or more mold pieces such as formation of a first plunger portion 152 and formation of second plunger portion 156 which are connected via a hinge to form the segmented plunger 150. The individual mold pieces may be assembled prior to loading of the segmented plunger 150 into the outer barrel 100 or assembled during the loading of the segmented plunger 150 into the outer barrel 100. The one or more hinges 154 may be formed by any means commonly known in the art. For example the hinge may be formed during injection molding of the segmented plunger 150 by creating a region where the material used to form the segmented plunger 150 is thinner than the surrounding material. This type of hinge is sometimes referred to as a “living hinge” in the art.

The segmented plunger 150 will also comprise a passageway, opening, channel or cavity 153 that will allow the removal string 340 of pledget 300 to pass through the segmented plunger 150 and the inner cavity of the outer barrel 100. Typically the passageway, opening, channel or cavity 153 will be formed in the first plunger portion 152 as will be described in greater detail below.

The segmented plunger 150 should also comprise the second plunger portion 156 that will be movably attached to the first plunger portion 152, preferably by a hinge and most preferably by a living hinge. The second plunger portion 156 will be outside the inner cavity of the outer barrel 100 prior to use of the tampon applicator and preferably mated, i.e., removably attached or clipped, to the outer barrel 100 as shown in FIG. 1B. The second plunger portion 156 should be sized and shaped to allow it to telescopically move or slide within the inner cavity of the outer barrel 100. In certain embodiments the second plunger portion 156 will be a hollow hemi-cylindrical or substantially hemi-cylindrical shape to allow the second plunger portion 156 to conform, mate or nest to the outer surface of the outer barrel 100 when the applicator is in a storage mode but also allow the second plunger portion 156 to slidably move into the inner cavity of the outer barrel 100 when the segmented plunger 150 in the use mode.

In one embodiment, the segmented plunger 150 will further comprise a locking portion 158. The locking portion 158 of the segmented plunger 150 will aid in orienting the first plunger portion 152 and the second plunger portion 156 into the correct or optimal positioning during the use mode as shown in FIG. 1E. For example the locking portion 158 of the segmented plunger 150 may prevent the second plunger portion 156 from moving past the desired longitudinal axis that would allow the segmented plunger 150 to easily slide into the inner cavity of outer barrel 100 and thereby push the pledget 300 from the insertion tip 110. The locking portion 158 may also provide additional rigidity and strength to the segmented plunger 150 which will again aid in easily sliding the segmented plunger 150 into the inner cavity of the outer barrel 100. The locking portion 158 may extend from the base of the first plunger portion 152, which is opposite the end of the first plunger portion 152 that will contact the base of the pledget 300. The locking portion 158 should also extend from the first plunger portion 152 beyond the terminal end of the outer barrel 100 that is opposite the insertion tip 110. In certain embodiments, the locking portion 158 is shaped to receive and/or mate with a portion of the outer surface of the second plunger portion 156.

Prior to use of the tampon applicator, a grip region 157 of the second plunger portion 156 is moved away from the insertion tip 110 in an arching or radial manner to a position that creates a continuous longitudinal axis between the first plunger portion 152 and the second plunger portion 156 as shown in FIGS. 1C and 1E. In certain embodiments, when the second plunger portion 156 is moved into the continuous longitudinal axis position, it will engage with the locking portion 158. In certain embodiments the engagement of the locking portion 158 and the second plunger portion 156 will comprise a snap, compression or friction interaction that will secure the segments of the segmented plunger 150 into a uniform and unitary structure. Once the second plunger portion 156 is engaged with the locking portion 158, the first plunger portion 152, the second plunger portion 156, and the locking portion 158 will form the segmented plunger 150 which should have sufficient rigidity and orientation with respect to the outer barrel 100 to allow the user to exert pressure on the grip region 157 of the segmented plunger 150 towards the insertion tip 110 of the outer barrel 100 and slide pledget 300 into contact with the petals 112 of the insertion tip 110 and push pledget 300 through the insertion tip 110 of the outer barrel 100 as shown in FIG. 1E.

The first plunger portion 152 may be made in a single or unitary piece or it may be made in multiple sections or portions that may be combined during assembly of the applicator or prior to assembly of the applicator.

As shown in FIG. 1F the first plunger portion 152 may comprise a first 252 and a second 352 lateral portion. The first 252 and second 352 lateral portions of the first plunger portion 152 may be made in a single or unitary piece, preferably with the locking portion 158 and second plunger portion 156. Alternatively, the first 252 and second 352 lateral portions of the first plunger portion 152 may be formed in separate and distinct pieces. The first 252 and second 352 lateral portions of the first plunger portion 152 may be joined together prior to insertion into the outer barrel 100. If the first 252 and second 352 lateral portions of the first plunger portion 152 are formed separately, they may be joined by any means known in the art such as adhesives, ultrasonic welding, melt adhesion, physical attachments means such as snaps, friction mating, compression mating, hooks, clips, bands, pins or any combination of the foregoing. Alternatively, the first 252 and second 352 lateral portions of the first plunger portion 152 may be formed in a single or unitary structure such as by injection molding. If the first 252 and second 352 lateral portions of the first plunger portion 152 are formed as a single or unitary structure, the first 252 and second 352 lateral portions of the first plunger portion 152 may be connected by a living hinge 652 that allows the first lateral portion 252 and second lateral portion 352 to be folded along a lateral arc or path and form the first plunger portion 152. After folding along the lateral arc, the first 252 and second 352 lateral portions of the first plunger portion 152 may be joined by any means known in the art such as adhesives, ultrasonic welding, melt adhesion, physical attachments means such as snaps, friction mating, compression mating, hooks, clips, bands, pins or any combination of the foregoing. As shown in FIG. 1F, the first 252 and second 352 lateral portions may be joined to form the first plunger portion 152 by a clip 800 located on the outer surface of the first lateral portion 252 that will interact with a locking projection 810 on the outer surface of the second lateral portion 352. The clip 800 may alternatively be located on the outer surface of the second lateral portion 352 and the locking projection 810 may be located on the outer surface of the first lateral portion 252.

As shown in FIG. 1G the first plunger portion 152 may comprise a first 452 and a second 552 vertical portion. The first 452 and second 552 vertical portions of the first plunger portion 152 may be made in a single or unitary piece, preferably with the locking portion 158 and second plunger portion 156. Alternatively, the first 452 and second 552 vertical portions of the first plunger portion 152 may be formed in separate and distinct pieces. The first 452 and second 552 vertical portions of the first plunger portion 152 may be joined together prior to insertion into the outer barrel 100. If the first 452 and second 552 vertical portions of the first plunger portion 152 are formed separately, they may be joined by any means known in the art such as adhesives, ultrasonic welding, melt adhesion, physical attachments means such as snaps, friction mating, compression mating, hooks, clips, bands, pins or any combination of the foregoing. Alternatively the first 452 and second 552 vertical portions of the first plunger portion 152 may be formed in a single or unitary structure such as by injection molding. If the first 452 and second 552 vertical portions of the first plunger portion 152 are formed as a single or unitary structure, the first 452 and second 452 vertical portions of the first plunger portion 152 may be on connected by a living hinge 752 that allows the first vertical portion 452 and second vertical portion 352 to be folded along a vertical arc or path and form the first plunger portion 152. After folding along the vertical arc, the first 452 and second 552 vertical portions of the first plunger portion 152 may be joined by any means known in the art such as adhesives, ultrasonic welding, melt adhesion, physical attachments means such as snaps, friction mating, compression mating, hooks, clips, bands, pins or any combination of the foregoing.

The first plunger portion 152 should contain a passageway 153 that will allow the passage of the removal string 340 through the first plunger portion 152 and through the outer barrel 100. In certain embodiments, the passageway 153 may be located in the first 252 and/or second 352 lateral portions or in the first 452 and/or second 552 vertical portions. Alternatively, the passageway 153 may be a tract, channel, groove or indentation on the outer surface of the first plunger portion 152 that will allow the removal string 340 to pass through the first plunger portion 152 and through the outer barrel 100 while also allowing the segmented plunger 150 to move freely within the inner cavity of the outer barrel 100. In certain embodiments, the removal string 340 passes through the first plunger portion 152 and is located on the inner surface 156a of the second plunger portion 156 as shown in FIGS. 1C and 1E, especially when the plunger is in a stored mode and a use mode. In certain embodiments the passageway 153 in the first plunger portion 152 is angled, tapered, sloped or conical so that the opening 153a closest to the base of the pledget 320 is wider or larger than the opening 153b that is closest to the second plunger portion 156. This angle, taper, slope or conical shape of passageway 153 from 153a to 153b aids in directing the removal string 340 toward the inner surface 156a of the second plunger portion 156.

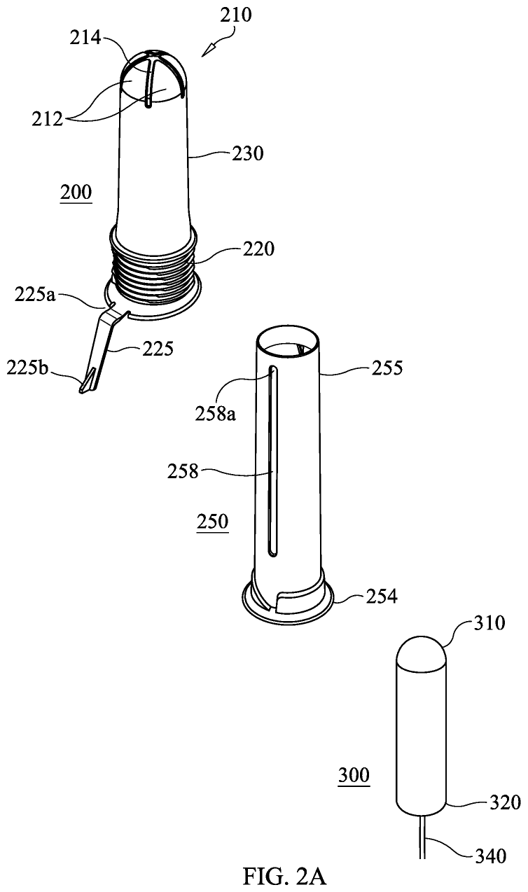

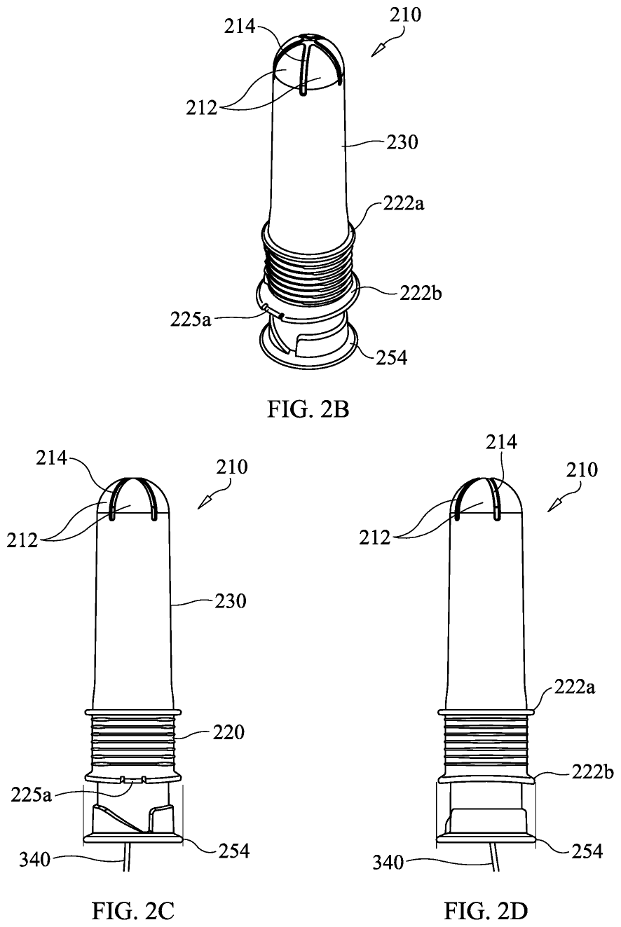

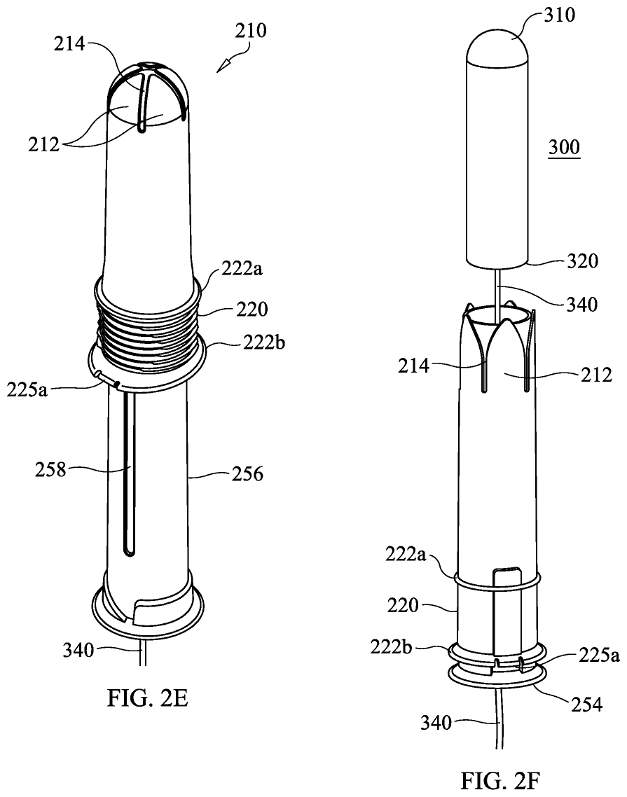

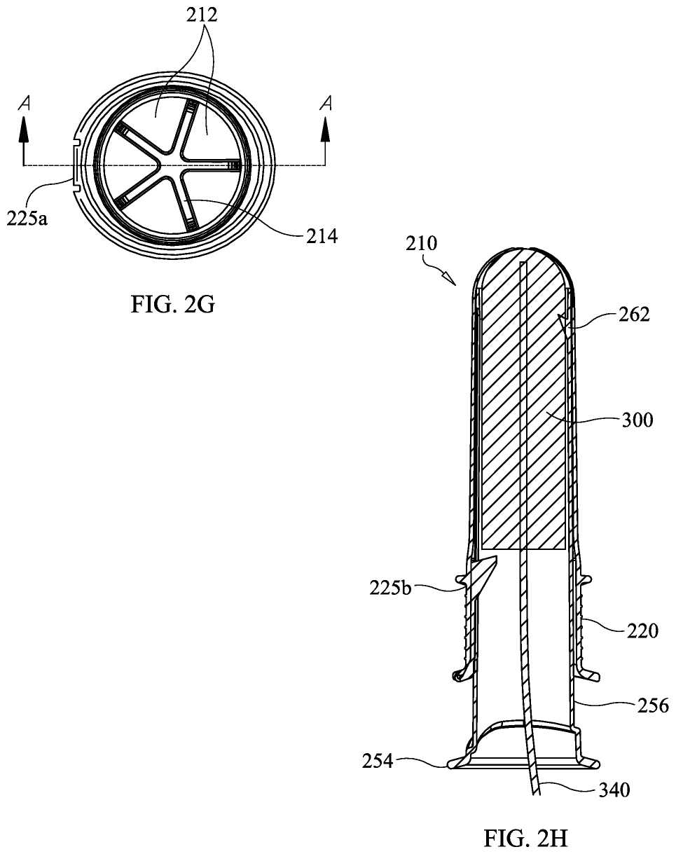

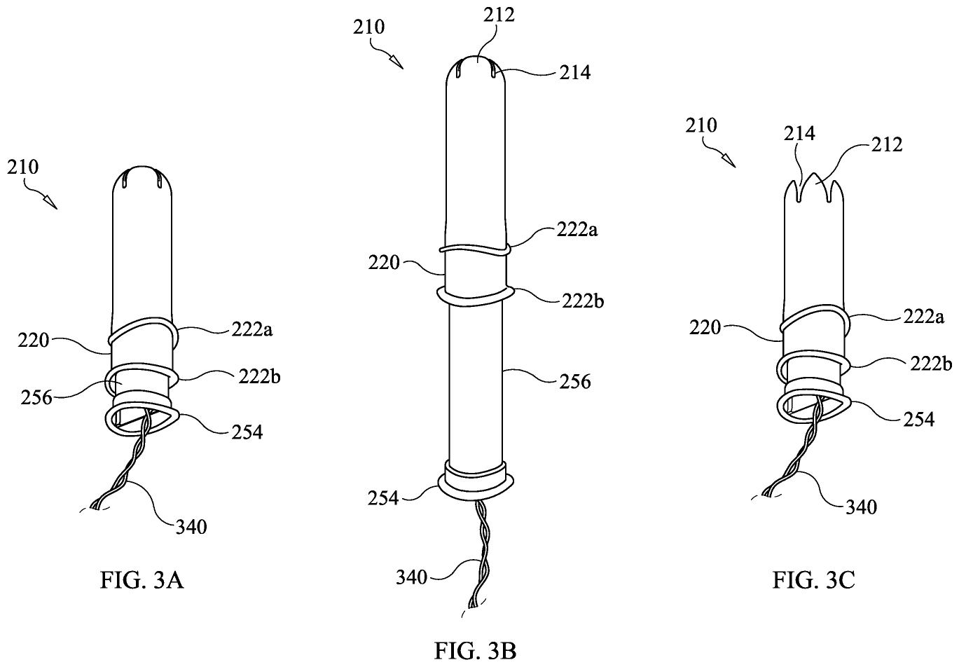

Referring now to FIGS. 2A to 2H and 3A to 3C which depict second embodiments of the present invention wherein the plunger is nested within the outer barrel prior to use. FIG. 2A shows the three primary components of the nested plunger embodiment of the present invention which comprises the outer barrel 200, pledget 300 and nested plunger 250. The outer barrel 200 comprises an insertion tip 210, a gripping region 220 that is opposite the insertion tip 210, a main body region 230 located between the insertion tip 210 and gripping region 220, an inner cavity that encases the pledget 300 and a substantial portion of the plunger 250.

The insertion tip 210 may be domed or hemispherical in shape or it may be tapered or bullet shape to allow easier and more comfortable insertion into the vaginal cavity during use as previously described. The insertion tip 210 comprises a plurality of petals 212 that are formed into the insertion tip and separated by a plurality of cuts, slits, or spacings 214. The petals 212 may be sized and shaped to provide protection to pledget 300 prior to and/or during insertion of the tampon applicator into the vaginal cavity for subsequent expulsion of pledget 300 into the vaginal cavity. The petals 212 may also be sized and shaped to protect the user from the rough surface of the pledget 300 during insertion. Embodiments of the present invention may comprise 3 to 8 petals, preferably 4 to 6 petals, and the dimensions of the petals are also previously described. In certain embodiments, the petals 212 are formed into the dome or tapered shape of the insertion tip 210 during the manufacture of the outer barrel 200 preferably during the injection molding of the outer barrel 200 and before pledget 300 is loaded into outer barrel 200. Alternatively, the dome or tapered shape of the insertion tip can be formed after initially forming outer barrel 200 and after pledget 300 has been loaded into the outer barrel 200. The performing of the dome or tapered shape of the insertion tip 210 during the manufacture of the outer barrel 200 and/or before the insertion of the pledget 300 into the outer barrel 200 is the preferred method because it allows for simpler manufacturing, i.e., less processing steps.

The gripping region 220 is located on the outer surface of the outer barrel 200 and opposite the insertion tip 210. The gripping region 220 is an area of the outer barrel 200 which will allow the user to grip or hold the tampon applicator during use. The gripping region 220 may comprise one or more structures to improve the users hold on the outer barrel 200 such as those previously described for the segment plunger embodiment. In one embodiment, the gripping region 220 comprises at least one, preferably at least two raised projections 222 that extend outwardly from the outer surface of the outer barrel 200 and define the gripping region 220 of the outer barrel 200. At least one of the raised projections 222a should be placed on the outer surface of the outer barrel 200 between the insertion tip 210 and the terminal end of the outer barrel 200 that is opposite the insertion tip 210 and preferably at a distance from the terminal end of the outer barrel 200 that is sufficient to accommodate a fingertip as previously described. In one embodiment, a second raised projection 222b may be employed at or near the terminal end of the outer barrel 200 that is opposite the insertion tip 210. In this embodiment first raised projection 222a and the second raised projection 222b define the gripping region 220. As shown in FIG. 2A to FIG. 2D the raised projections 222a and 222b extend outwardly from the outer barrel 200 in a continuous and uniform plane. In an alternative embodiment, as shown in FIG. 3A to 3C, the first raised projection 222a may be a continuous undulating or sinusoidal projection as previously described. In some embodiments, the terminal end of the outer barrel 200 that is opposite the insertion tip 210 and/or the raised lower projection 222b are a concave shape, i.e., curving inwardly toward the insertion tip 210 as previously described.

Attached to the terminal end of the outer barrel 200 that is opposite the insertion tip 210 and/or the raised lower projection 222b, is a retaining arm 225. When the tampon applicator is in an unassembled state, i.e., before the pledget 300 and plunger 250 are inserted into the inner cavity of the outer barrel 200, the retaining arm 225 extends from the terminal end of the outer barrel 200 that is opposite the insertion tip 210 and/or the raised lower projection 222b and is attached to the terminal end of the outer barrel 200 that is opposite the insertion tip 210 and/or the raised lower projection 222b by a hinge 225a, preferably a living hinge as described previously for the segmented plunger. The retaining arm 225 also comprises a holding and guiding projection 225b. In the assembled state, i.e., when the pledget 300 and plunger 250 are inserted into the inner cavity of the outer barrel 200, as shown on FIGS. 2B, 2C and 2D, the retaining arm 225 is located in the inner cavity of the outer barrel 200.

The pledget 300 is similar to the pledget described previously and comprises an insertion tip 310, a base 320 that is opposite the insertion tip 310 and a removal device or string 340. The pledget 300 is stored in the inner cavity of the outer barrel 200 and the inner cavity of the plunger 250 which will be described in greater detail below. In certain embodiments the pledget 300 is loaded into the inner cavity from the terminal end of the outer barrel 200 opposite the insertion tip 210. The loading of the pledget 300 preferably after the dome or tapered shape of the insertion tip 210 has been created and may occur prior to the loading of the plunger 250 into the outer barrel 200, concurrently with the loading of the plunger 250 into the outer barrel 200 or after the loading of the plunger 250 into the outer barrel 200. The loading of the pledget 300 through the terminal end of the outer barrel 200 with a preformed domed or tapered insertion tip shape also allows the inner surface of the outer barrel 200 to be formed without the need for retaining and/or support structures such as barbs or hooks in the insertion tip region of the outer barrel 200.

The plunger 250 comprises an insertion end 255, a push end 254, a body portion 256 located between the insertion end 255 and push end 254, a guide track or opening 258 and an inner cavity that extends longitudinally through the plunger 250. The plunger 250 has a size and dimension that allow it to slide along the longitudinal axis of the inner cavity of the outer barrel 200. As shown in FIG. 2H, the plunger 250 contains one or more retaining projections 262 that extend inwardly from the wall of the plunger 250 into the inner cavity of the plunger 250. The one or more retaining projections 262 are located near the insertion end 255 of the plunger 250. As also shown in FIG. 2H, when this embodiment of the present invention is assembled, retaining arm 225 is positioned in the inner cavity of the outer barrel 200 and the holding and guiding projection 225b of retaining arm 225 extends inwardly towards the inner cavity of the outer barrel 200 and the inner cavity of the plunger 250. The holding and guiding projection 225b will engage the guide track or opening 258 of plunger 250 and will guide the longitudinal movement of the plunger 250 within the inner cavity of the outer barrel 200. The holding and guiding projection 225b will also engage the base 320 of pledget 300 and prevent the movement of pledget 300 away from the insertion tip 210 of the outer barrel 200 when the nested plunger is moved from a compacted position to a ready to use position.

In one embodiment, when the nesting plunger embodiment is assembled, the plunger 250 with or without the pledget 300 is inserted into the terminal end of the outer barrel 200 opposite the insertion tip 210 and oriented so that the holding and guiding projection 225b engages the guide track or opening 258 of plunger 250. The plunger 250 is pushed into the outer barrel 200 until the insertion end 255 of plunger 250 engages the base of the petals 212 and/or the base of the openings 214 to create a nested or compacted position for the tampon applicator. The retaining arm 225 is moved into the inner cavity of the outer barrel 200 before or during the insertion of the plunger 250. Once the plunger 250 is in the nested or compacted position, the insertion tip 310 of pledget 300 is inserted into the push end 254 of plunger 250 and moved longitudinally through the inner cavity of plunger 250 until the insertion tip 310 of pledget 300 extends from the insertion end 255 of plunger 250 and preferably, but not necessarily, contacts petals 212 of the insertion tip 210 of outer barrel 200. The insertion tip 310 of pledget 300 may also be in contact with the petals 212 when the plunger is in the nested or compacted position. In certain embodiments, the plunger 250 may be inserted into the outer barrel 200 with the pledget 300. In this embodiment, the pledget 300 is inserted into the push end 254 of plunger 250, preferably, but not necessarily, with the insertion tip 310 of pledget 300 extending from the insertion end 255 of plunger 250, and the removal string 340 extending longitudinally from the base 320 of pledget 300 through the inner cavity of plunger 250 and extending rearwardly through the push end 254 of plunger 250.

When using the nesting plunger embodiment, the user will pull the push end 254 of plunger 250 longitudinally away from the insertion tip 210 of outer barrel 200 until the holding and guiding projection 225b engages the stop end 258a of guide track or opening 258 of plunger 250. The holding and guiding projection 225b will also be in contact with the base 320 of pledget 300 while the user pulls the plunger 250 rearwardly from outer barrel 200. The engagement of the holding and guiding projection 225b with the base 320 of pledget 300 will prevent the pledget 300 from moving rearwardly with the plunger 250 during the ready to use phase and help to maintain the insertion tip 310 of pledget 300 in contact with or in close proximity to the petals 212 of the insertion tip 210 of outer barrel 200. Once the plunger 250 has been pulled rearwardly so the holding and guiding projection 225b engages the stop end 258a of guide track or opening 258 and stops the rearward movement, the retaining projections 262 that depend inwardly into the inner cavity of plunger 250 should engage the base 320 of pledget 300. Once the plunger 250 has been pulled rearwardly and the retaining projections 262 engage the base 320 of pledget 300, the nesting plunger embodiment is in the loaded or ready to use position as shown in FIGS. 2E and 3B. From the loaded or ready to use position the user will hold the gripping region 220 of outer barrel 200 and exert pressure on the push end 254 of plunger 250 towards the insertion tip 210 of the outer barrel 200 and thereby slide pledget 300 through the insertion tip 210 of the outer barrel 200 as shown in FIGS. 2F and 3C.

In certain embodiments the holding and guiding projection 225b and the retaining projections 262 may be any shape or size that will allow the pledget 300 to be loaded into the tampon applicator without substantially damaging the pledget 300 but will also provide sufficient contact with the base 320 of pledget 300. In some embodiments, as shown in FIG. 2H, a cross sectional shape of holding and guiding projection 225b and a cross sectional shape of retaining projections 262 may be triangular or substantially triangular, wherein one leg of the triangle is parallel or substantially parallel with the longitudinal axis of the outer barrel 200 and/or the plunger 250, one leg or base of the triangle is perpendicular or substantially perpendicular to the longitudinal axis of the outer barrel 200 and plunger 250, and one leg of the triangle extends outwardly from the push end 254 of the plunger toward the insertion tip 255 of the plunger 250.

Typical dimensions for each of the outer barrel and plunger includes a length of from about 50 to about 100 millimeters, a diameter of from about 8 to about 16 millimeters, and a thickness of from about 0.4 to about 0.6 millimeters. Preferably, the diameter of the plunger is less than the diameter of the inner cavity of the outer barrel to allow for a telescopic arrangement of the two. Typical dimensions and arrangement of the petals are known in the art and described in numerous references such as U.S. Pat. Nos. 3,895,634; 6,432,075 and 6,773,422 which are incorporated herein by reference.

In addition to the benefits already noted for the above embodiments, the present invention will also exhibit the following improvements and benefits:
In the final ejection position, the gripping or push region of the plunger is at a greater distance from the grip region of the barrel, thereby avoiding gripping the applicator too tightly between the index finger, middle finger and thumb which can lead to difficulty in pushing the plunger, incomplete ejection of the pledget from the applicator and resulting in less chance of pinching the labia between the outer barrel and the plunger;
No need for barbs or projections at the tip of the inner cavity of the outer barrel which can interfere with plunger movement and pledget ejection and further allows for an easier manufacturing; and
The insertion tip of the plunger stops at the base of the petals to ensure proper release to the pledget.

The invention illustratively described herein suitably may be practiced in the absence of any element or elements, limitation or limitations which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A compact tampon applicator comprising:
(i) an outer barrel;
(ii) a pledget; and
(iii) a plunger;
wherein the plunger is a segmented plunger with a first portion of the plunger located inside the outer barrel near a gripping end of the outer barrel prior to use and a second portion of the plunger located outside the outer barrel prior to use; the first portion of the plunger has a pledget contact end that is inside the gripping end of the outer barrel and will contact the base of the pledget inside the outer barrel; the first portion of the plunger will also have a connection end that is opposite the pledget contact end and connected to the second portion of the plunger; the second portion of the plunger has a connection end that is moveably attached to the connection end of the first portion of the plunger and a push end that is opposite the connection end; prior to use, the push end of the second portion of the plunger is moved away from the insertion end of the outer barrel to a position that creates a continuous longitudinal axis between the first portion of the plunger and the second portion of the plunger with the second portion of the plunger extended away from the gripping end of the outer barrel.

2. The tampon applicator as defined in claim 1 wherein the segmented plunger comprises a locking portion that extends from connection end of the first portion of the plunger and that mates with the connection end of the second portion of the plunger when the second portion of the plunger is moved into the position creating a continuous longitudinal axis between the first portion of the plunger and the second portion of the plunger.

3. The tampon applicator as defined in claim 1 wherein the first portion of the plunger comprises a passageway that allows a removal string extending from the pledget to pass through the passageway and extend from the outer barrel of the applicator.

4. The tampon applicator as defined in claim 3 wherein passageway directs the removal string to the inner surface of the second portion of the plunger.

5. The tampon applicator as defined in claim 3 wherein the passageway is angled, tapered, sloped or conical so that the section of the passageway closest to the base of the pledget is larger than the section of the passageway closet to the second portion of the plunger.

6. The tampon applicator as defined in claim 1 segmented plunger is formed in a single injection molded piece.

7. The tampon application as defined in claim 1 wherein the segmented plunger is formed in multiple injection molded pieces and assembled prior to insertion into the outer barrel.
